(19) United States
(12) Patent Application Publication
(10) Pub. No.: US 2003/0018320 A1
(43) Pub. Date: Jan. 23, 2003

(54) Feeding probe for parenteral feeding of a patient, and its use

(76) Inventor: Rolf Rainer Scheu, Frankfurt (DE)

Correspondence Address: Pauley Petersen Kinne & Erickson, 2800 W. Higgins Road, Suite 365, Hoffman Estates, IL, 60195, US

(21) Appl. No.: 10/167,260

(22) Filed: Jun. 11, 2002

(30) Foreign Application Priority Data
Jun. 11, 2001 (DE) 101 28 094.7
Nov. 2, 2001 (DE) 101 54 043.4
May 17, 2002 (DE) 202 07 783.7

(51) Int. Cl. A61M 25/16

(52) U.S. Cl. 604/537; 604/516; 604/540

(57) ABSTRACT

A feeding probe for parenteral feeding of a patient. The feeding probe has a tube with a proximal end and a distal end and with a lumen passable in the tube between the proximal end and the distal end. In a region of the distal end of the tube, circumferential outlet openings are embodied for the discharge feeding solution introduced into the lumen of the tube via the proximal end. Between the circumferential outlet openings and the distal end of the tube, a guide portion free of openings is embodied, with a length that is greater than a diameter of the intestine into which the distal end of the feeding probe is introduced. A gastric probe is used in conjunction with the feeding probe for gastric decompression.

### BACKGROUND OF THE INVENTION

1. Field of the Invention

This invention relates to a feeding probe for a parenteral feeding of a patient, having a tube with a proximal end and a distal end and a lumen, which is continuous from the proximal end to the distal end, with a distal outlet opening of the lumen on the distal end of the tube, wherein outlet openings for the discharge of a feeding solution introduced into the lumen of the tube via the proximal end are embodied near the distal end of the tube and distributed over the circumference of the tube.

2. Description of Related Art

Feeding probes are inserted into the intestine for the sake of bypassing, relieving or sparing the stomach and are also known as jejunal probes. They are inserted into the patient through the nose, preferably endoscopically.

Such feeding probes for the parenteral feeding of a patient are known in many forms and are used for instance for postoperative feeding of the patient, with the feeding probe introduced into the patient by its distal end region that has the outlet openings, through the stomach into the intestine, so that an appropriate feeding solution and/or medication solution can be introduced via the proximal end, protruding from the patient's nose, of the feeding probe and can discharge directly into the intestine for feeding the patient, without there being any contact of the feeding solution with the gastric walls of the patient. Also, a patient can be given contrast medium so that an examination of the intestine can be performed.

Because of the placement of feeding probes, which is preferably done through a working conduit of an endoscope, these feeding probes have only extremely slight outside diameters, preferably only from 1.5 to 3.5 mm, equivalent to approximately 4 to 11 CH, but because the inside diameter for carrying the feeding solution, for instance, should be as large as possible, the wall thickness of such feeding probes is necessarily slight, being only 0.5 mm, for instance. Thus, there is one problem because the known feeding probes easily kink or become deflected out of their desired orientation.

Especially in patients in which reverse peristalsis occurs, where the contractions exerted by the intestine are oriented in the reverse direction, that is, toward the stomach, in the known feeding probes a diversion of the distal end in the direction of the stomach often occurs, causing it to kink, which causes a partial or even complete closure of the feeding probe and impairs or even prevents the delivery of the feeding solution. If because of the reverse peristalsis of the intestine, the distal end of the feeding probe is fed back as far as the inside of the stomach, then it is possible for solutions intended for the intestine to discharge from the feeding probe into the stomach, which is extraordinarily undesirable and can cause complications. As a rule, these events require the feeding probe to be removed and a new feeding probe to be put in place, which involves major effort and undesired stress on the patient.

The circumferential outlet openings on the tube that are absolutely necessary in the region of the distal end of the feeding probe, so that the feeding solution can discharge from the lumen inside the tube of the feeding probe, promote this undesired diversion and the associated kinking of the distal end, because these outlet openings intrinsically weaken the cross section of the tube and accordingly reduce the dimensionally stability and rigidity of the tube in this region.

U.S. Pat. No. 4,704,111 teaches a feeding probe in which the tube is thickened in the region of the distal end, compared to the region adjacent to the proximal end of the feeding probe. The distal end of the tube is closed, and the probe tip becomes heavier by additional weight. For the discharge of the feeding solution, circumferential outlet slits are embodied in the thickened region of the tube. The weight of the closed probe tip prevents the diversion and/or kinking of the feeding probe in the region of its distal end. However, feeding probes of this kind, with additional weights located in the distal end region, are complicated to manufacture and are also relatively ineffective, because of the small diameter of feeding probes and their small dimensions they have only a little additional weight, which experience teaches is no hindrance to diversion and kinking in the case of reverse intestinal peristalsis.

If the feeding probe remains in the intestine of a patient for a relatively long time, it is known to place a second probe, namely a gastric probe, in the stomach of the patient in order to drain gastric secretions and decompress the stomach. This gastric probe, which is not used to deliver feeding solution, is advanced into the patient's stomach through the nose as well, over the feeding probe already placed in the intestine, and is also called a nasogastric probe.

Gastric probes substantially comprise a tube made from a soft plastic hose; inert, physiologically harmless plastics are used as the material, examples being thermoplastic polyurethane elastomers or silicon rubbers or soft PVC. The surface of the gastric probe must be soft and supple, in order to prevent injuries to the mucus membranes as the gastric probe is introduced. Despite the soft, flexible plastic, the gastric probe must also have sufficient stability to prevent kinking as it is introduced into the stomach. In addition, the surface of the gastric probe must have a good sliding capability.

Since gastric probes are typically advanced through the nose of a patient into the stomach, the outer diameter of the gastric probe, of the tube, is limited by the nasal passage to a maximum of about 16 CH, which is equivalent to about 5 to 5.3 mm and represents the maximum size for comfort for the patient. If the gastric probe is placed in conjunction with a feeding probe, then the feeding probe is passed through the lumen of the gastric probe, or the tube probes are guided separately. That requires having the largest possible free inside diameter of the lumen of the gastric probe to accommodate the feeding probe inside it, and an interstice must also remain free between the feeding probe and the gastric probe in the lumen, an annular gap, to enable the drainage of gastric secretions through the lumen of the gastric probe despite the feeding probe placed inside it. Because feeding probes with a size of 9 CH are typically used, which is equivalent to an outside diameter of the feeding probe of about 3 mm, problems arise in embodying the gastric probe with an adequate wall thickness and stability against kinking, yet with the requisite softness and flexibility of the plastic used for the purpose.

For embodying a gastric probe for drainage and decompression that is to be used in conjunction with a feeding probe, it is desirable that: the gastric probe have the least possible wall thickness, in order to achieve the greatest possible inside diameter of the continuous lumen for drainage; the surface of the tube of the gastric probe be soft and supple, to avoid injuries to the mucus membranes; the feeding probe slide easily through the lumen of the gastric probe, with the use of additional lubricants to be maintained as slight as possible or to be avoided; the gastric probe be embodied as radiopaque; and the gastric probe be shear-stable, with sufficient softness and flexibility.

### SUMMARY OF THE INVENTION

It is one object of this invention to provide a feeding probe of the generic type but so that in the simplest possible way, a diversion or bending double of the distal end region thereof from reverse peristalsis of the intestine of the patient is reliably prevented, so that flow through the feeding probe cannot be disrupted or interrupted and a reverse motion of the feeding probe into the stomach cannot occur.

It is another object of this invention to combine the feeding probe with a gastric probe, so that in addition to the delivery of feeding solution and/or medication solution to the intestine of a patient, the stomach can also be drained and relieved.

According to this invention, beginning at the distal end and extending in the direction of the proximal end, the feeding probe is embodied with a guide portion. The guide portion has a length that is greater than the diameter of the patient's intestine into which the distal end of the tube of the feeding probe is introduced. The guide portion has a length of at least 30 mm. Outlet openings embodied on the circumference of the tube are disposed outside the guide portion.

According to this invention, it has been found that by embodying a guide portion that is free of openings and is located in the region of the distal end of the feeding probe, which is achieved by sufficient spacing apart of the circumferential outlet openings, which weaken the cross section of the tube, from the distal end thereof, the diversion and folding over or kinking of the distal end region of the feeding probe inside the intestine is reliably prevented, in an unexpected simple way. Specifically, if reverse peristalsis or unusual movements of the patient cause the distal end region of the feeding probe of this invention to deflect outward, then because of the greater length of the guide portion in comparison to the diameter of the intestine, the feeding probe can not be diverted in the direction opposite the introduction direction, because then the distal end region comes to rest prematurely on the intestinal walls, thus preventing any further diversion motion.

According to this invention, the usual expected diameter of a patient's intestine into which the distal end region of the feeding probe is introduced is about 20 to 40 mm. Thus, according to this invention the length of the guide portion, achieved by spacing apart the circumferential outlet openings from the distal end of the tube by at least 30 mm and preferably 50 mm or more, prevents the diversion of the distal end region, for instance caused by a reverse intestinal peristalsis.

Reliable prevention of the diversion can be achieved with feeding probes of this invention in which the guide portion has a length L and thus a suitable spacing of the outlet openings from the distal end of the tube is embodied such that the ratio D:L of at least 1:1.2 is achieved, where D is the diameter of the patient's intestine. Since the length L is at least 1.2 times the diameter D, upon a deflection the guide portion of the tube is pressed against the intestinal wall, thus preventing the tube from being diverted or even bent double.

In practice, feeding probes of various sizes can be embodied according to this invention, with variations of the spacing L to adapt to various intended uses and patients, for example size, weight, age and the like. Preferably, the guide portion of the feeding probe has a length of 40 to 60 mm. The outlet openings located closest to the distal end of the tube of the feeding probe on the circumference of the tube then have a minimum spacing from the distal end of the tube of 30 mm and preferably 40 to 60 mm.

A further substantial advantage of the feeding probe of this invention is that it has no additional weights in the region of the distal end, making it possible to place a distal outlet opening in the tube that extends in the longitudinal axis thereof, so that even at the frontmost tip, in the insertion direction, of the feeding probe of this invention, the feeding solution can discharge from the lumen, unhindered. The feeding probe of this invention may have a tube of constant outer diameter over its length, making it shear-stable as well.

The distal outlet opening has the same diameter as the lumen embodied in the tube and thus causes no narrowing in the outlet direction. Thus, even medications that are insoluble in liquid that are to be administered through the feeding probe of this invention cannot be trapped in the lumen, and thus neither clogging of the feeding probe nor a loss of the active ingredient to be given to the patient can occur.

The high degree of security of the feeding probe of this invention against diversion or bending double and being moved rearward into the stomach can be further enhanced if the tube is made from an elastic material with high restoring force, for example of thermoplastic and/or elastomeric plastics, thermoplastic elastomers (TPE) and elastomerlike thermoplastics, with a Shore hardness A in accordance with ASTM D 2240 of less than or equal to 90, for instance on the basis of a polyurethane elastomer. The high restoring forces of the tube, upon deflection of the distal end region of the tube, in the event of reverse intestinal peristalsis, for instance, counteract the deflection and reinforce the restoration of the tube to its original, desired position after every deflection that occurs. Still other suitable plastics with adequate restoring force can also be used to construct the tube. Suitable plastics according to this invention can have either a single-layer or a multi-layer structure of a single material, or a combination of materials can be used, for instance multi-layer plastic composites of different plastics made by coextrusion.

According to this invention, the feeding probe may be radiopaque, and a passable embodiment of the tube is preferred. Thus, the tube is preferably made from a composition which for 100 parts by weight of the soft, flexible plastic contains from 10 to 15 parts by weight of a radiopaque medium, such as barium sulfate. This gives the tube an opaque color, and in a known manner it can have a length and depth marking in ink on its outside.

To increase the slidability of the feeding probe, for instance inside a gastric probe, the surface of the feeding probe may be microscopically rough. Thus a cushion of air and liquid can form that promotes sliding and counteracts adhesion. For simultaneous parenteral feeding and gastric decompression of a patient, the feeding probe according to this invention can be used with a gastric probe for draining gastric secretions and decompressing the patient's stomach. As the gastric probe, a probe of the kind that has a tube with a continuous lumen from the proximal end to the distal end and also has drainage holes emerging laterally from the tube of the gastric probe should be used. The feeding probe can be passed through the lumen of the tube of the gastric probe, forming an annular gap between the surface of the feeding probe and the surface that defines the lumen of the gastric probe.

The conditions for the combined use of a feeding probe of this invention with a gastric probe are achieved in a way that is especially comfortable to the patient with a gastric probe whose tube has a multi-layer structure. According to this invention, the gastric probe is slipped over the feeding probe and has a multi-layer tube, with an outer layer of a soft, flexible plastic and an inner layer of a harder plastic than the plastic comprising the outer layer, and the inner layer ends before the distal end of the gastric probe inside the passable lumen of the gastric probe, and the plastic used for the inner layer has a good sliding property and anti-adhesion property relative to the plastic from which the feeding probe is made.

Preferably, coextrudable plastics that are compatible with one another are used to produce a two-layer tube for the gastric probe.

The mutually compatible extruded plastics for the outer layer and the inner layer should have good adhesion to one another.

For the outer layer, conventional plastics used for gastric probes are preferably used, especially thermoplastic and/or elastomeric plastics, thermoplastic elastomers, and elastomerlike thermoplastics, preferably with a Shore hardness A in accordance with ASTM D 2240 of less than or equal to 90. Especially suitable soft, flexible materials for the outer layer of the tube are, for example, thermoplastic polyurethane elastomers, which are polyadducts of polyisocyanates and polyols; of these, thermoplastic polyurethane elastomers based on polyetherurethanes with a Shore hardness A preferably in the range between 70 and 90 are preferred.

For the inner layer, a hard, rigid plastic with a Shore hardness D in accordance with ASTM 2240 greater than 45, and preferably greater than 55, is preferably used. Once again, physiologically harmless plastics that are compatible with the elastomer used for the outer layer of the tube can be considered. According to one embodiment of this invention, a copoly(etheresteramide), also called a polyether block amide, is used for the inner layer and has a Shore hardness D of 55 to 80, preferably a Shore hardness D of greater than 60.

The hose-like outer layer of the tube of the gastric probe comprising an elastomeric plastic is soft and by itself is unstable and not kink-proof and it is stabilized by the coextruded inner layer of hard plastic, which is very thin, so that with the two-layer embodiment of the tube, a shear-stable, yet soft and flexible gastric probe is obtained. The thickness of the hard inner layer of the tube of the gastric probe should be less than 0.020 mm, preferably less than 0.015 mm, and especially preferably 0.01 mm or less. Because of the stabilizing effect of the inner layer of the tube of the gastric probe, it is possible to keep the total wall thickness of the tube thinner. Because of the hard inner layer of the tube, it is possible for the total thickness of the multi-layer tube of this invention, relative to the thickness of a single-layer tube of a soft, flexible plastic alone, to be reduced to a minimum, and thus to increase the inside diameter of the lumen of the gastric probe and thus also enlarge the drainage lumen.

In the selection of a suitable hard plastic for embodying the inner layer of the multi-layer tube for the gastric probe, a plastic that has a good sliding property and good antiadhesion property is also preferably used, to enable easy sliding of the feeding probe through the lumen of the gastric probe. A good sliding property and good antiadhesion properties are also attained by using polyether block amides.

Depending on the selection of materials for the outer layer and the inner layer of the multi-layer tube of the feeding probe, however, the inner layer must always be relatively thin. The thickness of the inner layer in proportion to the thickness of the outer layer of the tube ranges from 1:20 to 1:80.

According to this invention, in a gastric probe with a multi-layer tube according to this invention with an outside diameter corresponding for instance to 16 CH, it is possible to embody the thickness of the outer layer as between 0.35 and 0.45 mm and the thickness of the inner layer in the range from 0.015 to 0.006 mm. Thus according to this invention, a gastric probe with a multi-layer tube is obtained with an outer diameter corresponding for instance to 16 CH with a continuous lumen that has an inside diameter of at least 4 mm. Because feeding probes typically have a size of 9 CH with an outer diameter of 3.0 mm, it is possible to create an adequately large interstice between a feeding probe located inside the gastric probe and the inner wall of the tube of the gastric probe.

To avoid lesions in the stomach from the interface between the hard inner layer and the soft outer layer located at the distal tube end, which according to this invention is in multiple layers, the distal end of the multi-layer tube can be equipped with a soft, atraumatic radiopaque probe tip. The probe tip is preferably formed from an especially soft hoselike portion with a continuous lumen. Preferably the probe tip is of the same soft, flexible plastic as the outer layer of the tube, or a compatible plastic with a Shore hardness A that is approximately equivalent to the hardness of the plastic comprising the outer layer of the tube. The inside diameter of the lumen of the tube and the inside diameter of the lumen of the probe tip is preferably equivalent to one another.

In order to make the gastric probe radiopaque, according to this invention, only the probe tip is made radiopaque. For instance, the probe tip itself can be embodied as fully radiopaque, with from 10 to 15 parts by weight of a radiopaque medium, such as barium sulfate, contained in the probe tip per 100 parts by weight of the soft, flexible plastic. The probe tip preferably has a length of from 10 to 30 mm and is joined to the distal end of the tube so that it firmly adheres, for instance by being welded to it.

For enhanced drainage action, the tube of the gastric probe, in the region adjoining the probe tip, may have drainage holes that emerge laterally and that are preferably distributed over a region of a length of the tube of up to 250 mm, beginning at the probe tip.

The gastric probe is preferably embodied so that a Y connection can be connected to the proximal end of the tube. The straight leg of the Y connector provides passage to the feeding probe, while the lateral funnel attachment of the Y connector is used to attach a suction hose for draining the stomach through the gastric probe, and thus acts as the lumen of the tube of the gastric probe.

Conversely, the proximal end of the tube can be connected to an adapter for connection to a source of feeding solution or medication solution.

If a patient is given a gastric probe according to this invention, with a feeding probe located inside it, it is possible to supply a feeding solution to the intestine continuously through the feeding probe and simultaneously, by continuous or intermittent suction or drainage by gravity, via the remaining lumen of the tube of the gastric probe, to perform a pressure relief of the stomach. The feeding probe is located inside the gastric probe. Between the outer diameter of the feeding probe, which is typically 9 CH, and the inner diameter of the lumen of the gastric probe of this invention, which is 16 CH, there is an adequately large interstice, in the form of an annular gap with a gap width of at least 0.5 mm, which enables a pressure relief of the stomach via the lateral drainage holes of the tube of the gastric probe.

In one embodiment of this invention, for a feeding probe 9 CH in size and a length of at least 2 m, a gastric probe 16 CH in size with a length of at least 1 m that can be inserted via the feeding probe is provided, and the continuous lumen of the gastric probe has an inside diameter of at least 4 mm.

To enable particularly good sliding of the feeding probe inside the gastric probe, the surface of the feeding probe can have a microscopic roughness. As a result, an air cushion/liquid cushion is formed, and an adhesion of the surface of the feeding probe to the inner layer of the multi-layer tube of the gastric probe is avoided, and the slidability is improved considerably. According to this invention, a gastric probe embodied as a gastric decompression probe is proposed, which has a length of at least 1 m and is shear-stable by embodiment of the tube in multiple layers, with a soft, flexible outer layer of an elastomer and with an inner layer, joined to the outer layer by coextrusion, of a hard plastic. The inner layer ends before the distal end of the gastric probe inside the continuous lumen. The gastric probe of this invention has an extra-soft, radiopaque distal end in the form of a probe tip. The gastric probe according to this invention, which is soft yet shear-stable, is particularly well suited for use with a feeding probe inside it, preferably a nasojejunal feeding probe.

### BRIEF DESCRIPTION OF THE DRAWINGS

Further details and embodiments of a feeding probe according to this invention is described in view of the drawings that show one embodiment, wherein:

FIG. 1 is a front view of a feeding probe of this invention;

FIG. 2 shows a schematic, enlarged view of a location of the distal end region of the feeding probe of this invention, inside an intestine of a patient;

FIG. 3 shows the detail E in FIG. 2, in an enlarged partial sectional view;

FIG. 4 shows a schematic partial view of a production of a feeding probe of this invention, near the distal end thereof;

FIG. 5 shows a schematic front view of a gastric probe with a connector;

FIG. 6 shows an enlarged detail along the section line H-H of FIG. 5;

FIG. 7 shows an enlarged detail of the gastric probe of FIG. 6, with a feeding probe located inside it; and

FIG. 8 shows a schematic view of a feeding probe with a gastric probe for gastric decompression.

### DESCRIPTION OF PREFERRED EMBODIMENTS

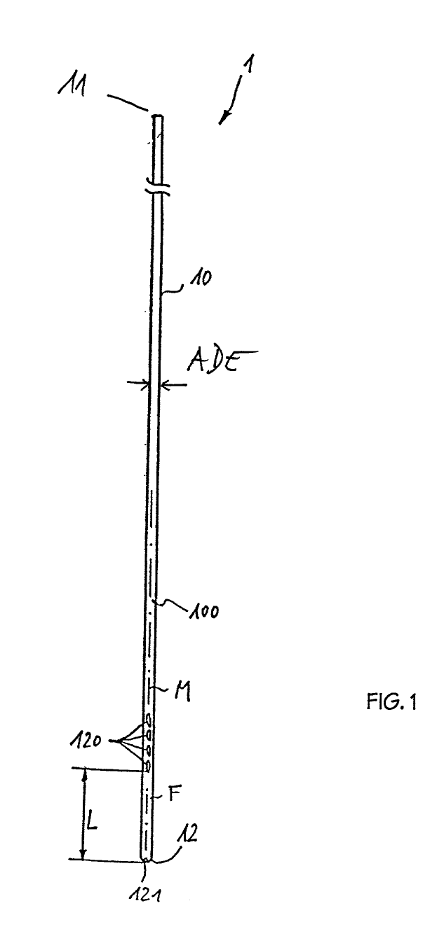

From FIG. 1, a feeding probe 1 for parenteral feeding of a patient is shown, which includes a tube 10 with a lumen 100 slidably mounted in it. The tube 10 of the feeding probe 1 extends from a proximal end 11 to a distal end 12, and the continuous lumen 100 is passable from the proximal end 11 to the distal end 12.

The outside diameter ADE of the tube 10 is constant and is approximately 3 mm, for a feeding probe that is 9 CH in size. The length of the feeding probe can be up to 3 m.

An adapter can be connected in airtight fashion to the proximal end 11 and is connectable to commercially available containers for feeding solutions, so that feeding solution can pass through the adapter and the proximal end 11 of the tube 10 to reach its lumen 100. From this lumen 100, the feeding solution flows out again via outlet openings 120, located circumferentially in the region of the distal end 12 of the tube 10, and a distal outlet opening 121 located in the direction of the longitudinal axis M of the tube 10. The circumferential outlet openings 120 are distributed over the circumference of the tube, approximately at a regular axial spacing from one another, for example every 10 mm.

Each outlet opening 120, of which there are for instance six, has a mean diameter of 2 to 2.5 mm, for a feeding probe 9 CH in size.

With the aid of the feeding probe 1, it is possible to feed a patient parenterally or for instance to administer a contrast agent.

Thus, the tube 10 is introduced with its distal end region 12 leading either orally or nasally in a manner known per se, optionally with the aid of an endoscope, through the patient's stomach and on into the intestine, so that the distal end 12, including the outlet openings 120, 121 located in this region rest inside the patient's intestine. The feeding probe 1 then protrudes out of the patient's mouth or nasal opening by its proximal end region 11, so that for instance from a container connected to the proximal end 11, a feeding solution can be administered directly into the intestine of the patient, for nourishment.

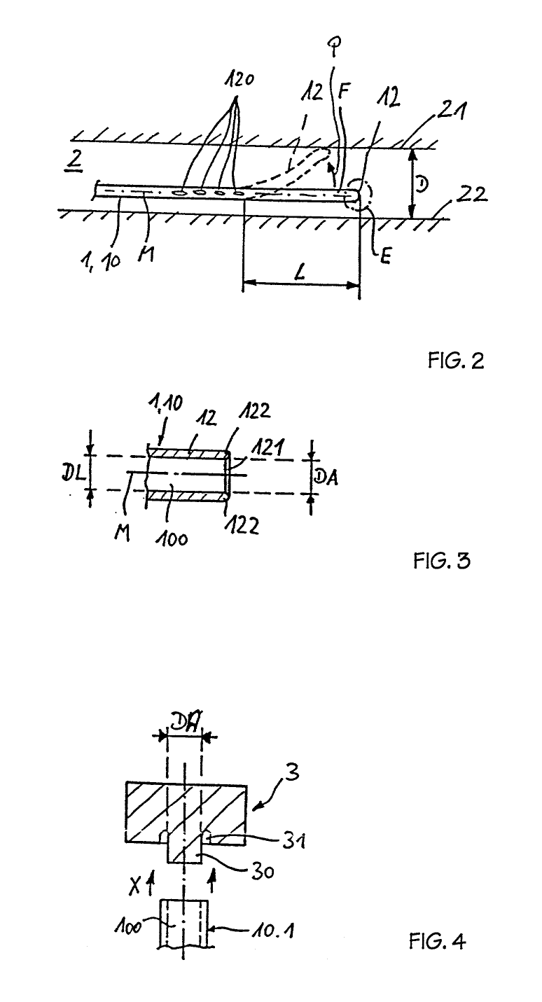

FIG. 2, in an enlarged schematic view, shows the location of the distal end region 12 inside the intestine 2 of the patient, for feeding the patient as described above through the feeding probe 1.

One essential characteristic of the feeding probe 1 is that the circumferential outlet openings 120, by way of which the feeding solution emerges from the lumen 100 of the tube 10 into the intestine, are disposed at a minimum spacing L from the distal end 12 of the tube 10 that is greater than the diameter D of the intestine 2, in which the distal end 12 of the feeding probe 1 comes to rest, of the patient to be fed. In this way, a guide portion F embodied with the length L is embodied without lateral outlet openings between the outlet openings 120 and the distal end 12. The ratio of the intestinal diameter D to the spacing L of the feeding probe is for instance 1:1.3, and as a concrete example the guide portion F can have a length L of approximately 40 mm.

The circumferential outlet openings 120 necessarily disposed in the tube 10 dictate a significant decrease in the stiffness of the tube 10 and therefore function like joints and enable the guide portion F disposed upstream of them to be deflected out of the desired orientation in the direction of the longitudinal axis M indicated by the arrow P, and this can be caused in particular by reverse peristalsis of the patient's intestine 2.

However, because the guide portion F is embodied with a length L greater than the diameter D of the intestine 2, its maximum deflection from the course of the longitudinal axis M is limited by its application to the intestinal wall 21 as shown by dashed lines in FIG. 2, so that the distal end 12 is reliably prevented from bending completely double in the direction of the proximal end 11 and thus toward the patient's stomach, and an at least partial closure of the feeding probe is thus reliably prevented. Thus, even reverse peristalsis of the patient cannot cause the feeding probe 1 to bend double and cannot cause the distal end 12 to return to the inside of the patient's stomach.

By selecting a suitable material for the tube 10 of the feeding probe 1, with a high elastic restoring force, it is possible not only to limit the deflection until contact with the wall 21 of the intestine 2 but also to accomplish a return of the guide portion F with the distal end 12 to the desired original location in the direction of the longitudinal axis M, which further enhances the security against bending double, closure and return of the distal end 12 from the desired location back into the patient's stomach. An elastic plastic material of this kind with a high restoring force can be formed for example of a suitable polyurethane, which can be quite tolerable to the patient and can be selected accordingly by one skilled in the art.

The number and disposition of the outlet openings 120 are also oriented to the requisite cross sections and can be selected accordingly by one skilled in the art. Only the minimum spacing 11 from the distal end 12 for embodying the guide portion F need be considered, for reliably preventing deflection and/or bending double of the distal end 12.

The distal outlet opening 121, located at the distal end 12 of the tube 10 in the direction of the longitudinal axis M, is embodied, as the enlarged view in FIG. 3 shows, with a diameter DA, which is equivalent to the diameter DL of the lumen 100 in the tube 10. Viewed in the direction of the longitudinal axis M of the tube 10, the outlet opening 121 therefore causes no narrowing of the lumen 100, so that medications that are insoluble in liquid and are carried through the lumen cannot be trapped there. Stopping up of the feeding probe with a loss of active ingredients is thus effectively counteracted.

Furthermore, the peripheral regions 122 of the tube 10 that surround the distal outlet opening 121 are embodied in rounded fashion, to avoid injuries to the patient's tissue.

This embodiment of the distal outlet opening 121 and of the peripheral regions 122 surrounding it is as shown schematically in FIG. 4, achieved by a tool 3 during the production of the feeding probe 1. First, a hose segment 10.1 that will form the later tube 10 is cut to the proper length in a manner known per se, and next, one end of it is pressed against the heated tool 3 in the direction of the arrow X, to form the distal end region 12.

The tool 3 has an arbor 30, which is introduced into the end of the lumen 100 in order to make the distal outlet opening 121 having the diameter DA. Thus, the arbor 30 has the desired diameter DA.

Extending all the way around the arbor 30, a groove 31 is provided that is rounded and that reshapes the originally sharp cut edges of the tube 10 into the desired rounded peripheral regions 122, as shown in FIG. 3.

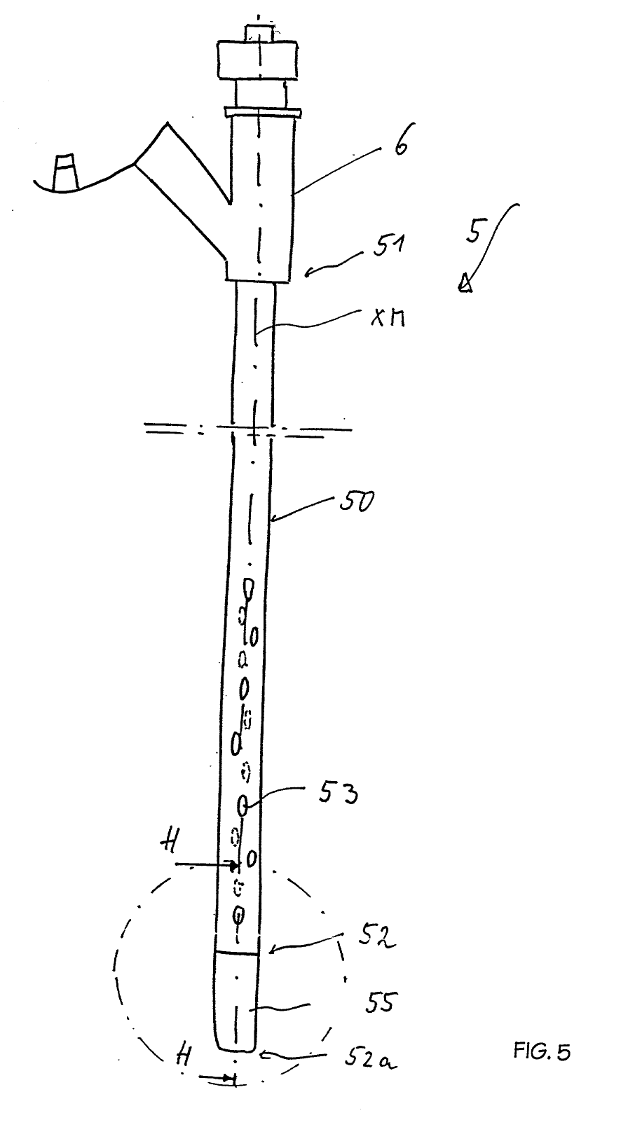

In FIG. 5, a gastric probe 5 is shown in an elevation view, with a tube 50 with a lumen that is continuous from the proximal end 51 to the distal end 52, and with a hoselike probe tip 55 adjoining the distal end 52 of the tube, the probe tip likewise having a passable lumen as far as the distal end 52a of the gastric probe 5. In the region adjoining the probe tip 55, the tube 50 has lateral drainage holes 53, holes that emerge laterally from the tube jacket. The drainage holes 53 are spaced apart equally from one another on the tube in the axial extent XM of the gastric probe or tube 50 and are offset over the circumference. A Y connector 6 is mounted on the proximal end 51 of the tube 50. The gastric probe should have a soft upper layer and surface, in order to avoid injuries. The upper layer/surface of the tube should be smooth upon contact with water or moisture, upon introduction into a patient. Furthermore, the tube of the gastric probe should have such high strength and stability that while it is bendable, it will not kink and the lumen will not be closed. At the same time, the wall thickness of the tube should be as small as possible, in order to gain a wide diameter for the continuous lumen, so that a feeding probe 1, for instance in accordance with FIG. 14 can be passed through it with play.

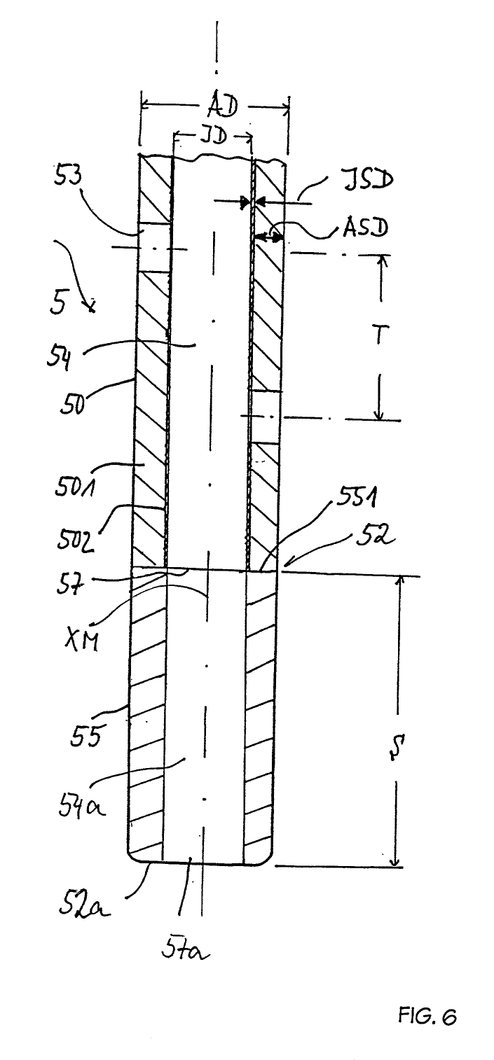

In FIG. 6, the gastric probe is shown in fragmentary form and in longitudinal section in the region of the tube 50 and the adjoining probe tip 55. The tube is in two layers, with a relatively thick outer layer 501 of a soft, flexible material, in particular a plastic with elastomeric and/or thermoplastic properties, and a very thin inner layer 502 of a hard, rigid plastic that is bonded with firm adhesion to the outer layer. The tube 50 is preferably produced by co-extrusion of the outer layer 501 and inner layer 502. Mutually compatible plastics of different hardness are used that are easy to extrude and that adhere to one another. If for the inner layer 502 a hard plastic is selected that does not enter into good adhesion to the material of the outer layer 501 in the course of the co-extrusion, it is possible to introduce an intermediate layer, as a third layer and adhesion promoter layer, between outer layer 501 and the inner layer 502 by co-extrusion.

The tube 50, constructed in two layers in the exemplary embodiment, of the gastric probe 5 has the lumen that is continuous from the proximal end to the distal end 52. The interface of the tube 50 at the outlet opening 57 on the distal end 52 is likewise hard, because of the hard inner layer 502 that is open here, and thus lesions could occur in the vulnerable mucus membranes of a patient. Thus, a soft, flexible probe tip 55 that has the same outer diameter AD as the tube 50 and the corresponding inner diameter ID of the lumen 54 of the tube is joined to the distal end 52 of the multi-layer tube 50. The probe tip also has the continuous lumen 54a, which discharges into the outlet opening 57a on the distal end 52a of the gastric probe. The probe tip has a length S, preferably of between 10 and 30 mm, for a total length of the gastric probe that is typically more than 1 m. The probe tip 55 is preferably embodied as continuously radiopaque. The probe tip 55 is preferably produced from the same soft, flexible plastic, or a plastic with corresponding properties, as the outer layer 501 of the tube 50. The tube 50 is produced by coextrusion of a corresponding hose and then cut to the particular desired length of gastric probe. The probe tip 55 is likewise produced in the form of an endless hose, for instance from a plastic filled with a radiopaque medium, such as barium sulfate, and then the individual probe tips are cut to length from the endless hose. The probe tip 55, given identical or compatible plastics for the outer layer 502 of the tube, can be welded directly to the distal end 52 of the tube. The weld seam 55 is shown in suggested fashion. The probe tip 55 is rounded on the distal end.

In the region adjoining the probe tip 55, the laterally emerging drainage holes 53 are disposed in the tube 50. For instance, from sixteen to twenty-two drainage holes are disposed at a spacing T of 10 mm, on opposite sides of the tube, and each drainage hole has a mean hole diameter of about 2.5 mm.

The outer diameter AD of the gastric probe or tube 50, for a size of 16 CH, is approximately 5 to 5.3 mm. With the two-layer structure of the tube according to the invention, an outer layer 501 of a soft, flexible plastic with a Shore hardness A of 80, for instance, and a thickness ASD of 0.4 mm is provided. As the inner layer 502, a hard plastic, such as a polyether block amide, with a Shore hardness D of 70 and a thickness ISD of 0.01 mm is provided. This yields an inside diameter ID of the lumen 54 of at least 4.18 mm. Thus an adequately wide lumen 54 is formed in the gastric probe 5, so that a feeding probe with a size of 9 CH and an outer diameter ADE of 3 mm can be passed with an adequate interstice and adequate play through the gastric probe and the tube 50.

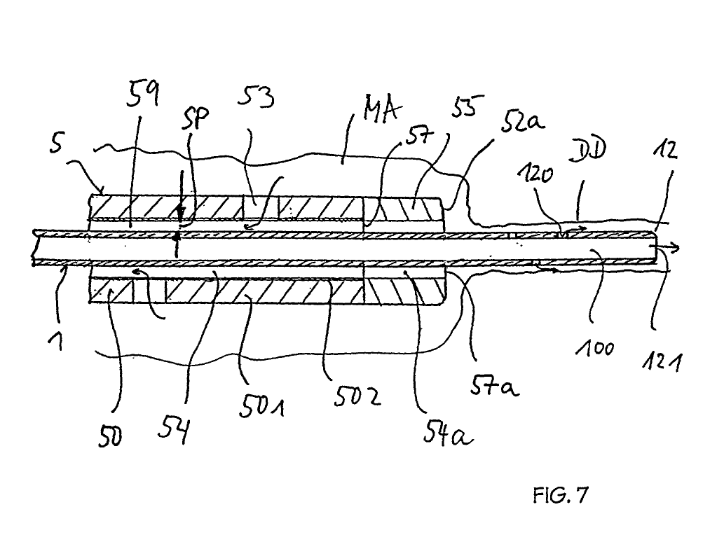

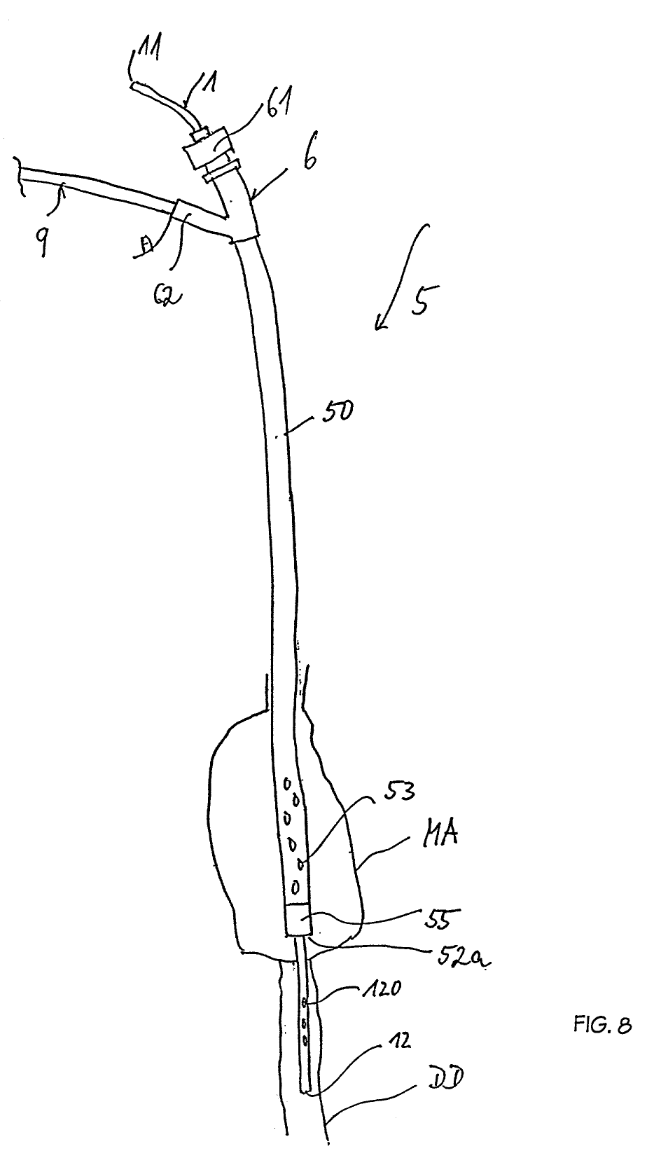

In FIG. 7, the feeding probe 1 introduced into an intestine DD of a patient is shown in fragmentary form; the gastric probe 5 is placed over it for the sake of drainage of decompression of the stomach MA of the patient. The feeding probe 1 is passed on the inside through the lumen 54 and 54a of the gastric probe 5 until it is inside the intestine DD. The feeding solution, which is supplied through the lumen 100 of the feeding probe 1, can emerge into the intestine DD through the outlet openings 120 and the distal outlet opening 121 of the feeding probe 1. The feeding probe 1 is introduced through the lumen 54, 54a of the gastric probe 5, leaving an annular gap space 59 having the width of the annular gap SP. The annular gap SP is calculated as the difference between the inner diameter ID of the lumen 54 of the tube 50 and the outer diameter of the feeding probe 1. Through the lateral drainage holes 53 of the tube 50, it is possible to drain the gastric secretions in the direction of the arrow and to decompress the stomach. The gastric probe used as shown in FIG. 7 is also called a gastric decompression probe.

FIG. 8 again shows a schematic total view of the gastric probe and the feeding probe in use for parenteral feeding and for gastric decompression. The feeding probe 1 is typically introduced nasally and has a size of 9 CH, for instance. It is combined with the gastric probe 5, which is 16 CH in size, for parenteral feeding and simultaneous relief of the stomach. The gastric probe has a probe tip 55, embodied as radiopaque, with small proportions of barium sulfate and is also equipped with a scale, not shown, on the tube in ink. For the connections, adapters and the connector 6 are provided. The feeding probe 1 is fabricated, for example, from a thermoplastic polyether urethane, as is the outer layer of the tube 50 of the gastric probe 5. For the hard inner layer of the gastric probe 5, a polyether block amide is for instance provided. The gastric probe is 125 cm long, for instance, and the feeding probe is 270 cm long; the additional equipage includes the Y connector 6 and also an adapter, not shown in detail, that is suitable for connecting the feeding probe 1 to a container or the like.

The feeding probe 1 is first introduced into the intestine of a patient; next, the gastric probe 5 is advanced over the feeding probe into the stomach. The correct location of both probes 1, 5 is monitored and ascertained with the aid of the printed depth markings on the probes 1, 5 or radiologically, by means of appropriate radiopaque regions of the probes. The proximal end 11 of the feeding probe 1, which protrudes from the connector 6 at its straight outlet 61, is connected to an adapter, not shown, and attached in airtight fashion. It is now possible to begin the feeding therapy or administration of medications through the feeding probe 1 introduced into the intestine DD. The nutrient solution can discharge through the outlet openings 120 of the feeding probe and the central outlet opening on the distal end 12 into the intestine DD. The straight connection 61 of the Y connector 6 serves the purpose of feeding through the probe or administering medications. The suction line 9 for extracting gastric secretions through the lumen 54, 54a of the gastric probe 5 is connected to the gastric lateral connection 62 of the connector 6, which is secured to the gastric probe 5. The gastric probe 5 is distinguished by a soft, atraumatic and radiopaque distal end 55. In ongoing feeding, continuous or intermittent suction or drainage by gravity can provide pressure relief to the stomach via the laterally offset drainage holes 53 of the gastric probe. Between the outer diameter of the feeding probe 1, which for instance is 9 CH in size, and the inside diameter of the gastric probe, which for instance is 16 CH in size, also called a gastric decompression probe, there is a large enough interstice to enable pressure relief via the lateral drainage holes of the gastric probe as well as the flow of gastric secretions through it in the direction of the arrow upon suction extraction.

German Patent Reference Nos. 101 28 094.7, 101 54 043.4, and 202 07 783.7, are the priority documents corresponding to this invention, and their teachings are incorporated, by reference, into this specification.

1. In a feeding probe for parenteral feeding of a patient, having a tube (10) with a proximal end (11) and a distal end (12) and a lumen (100) which is continuous from the proximal end (11) to the distal end (12), with a distal outlet opening (121) of the lumen (100) on the distal end (12) of the tube (10), wherein outlet openings (120) for discharge of a feeding solution introduced into the lumen (100) of the tube (10) via the proximal end (11) are embodied in a region of the distal end (12) of the tube (10) and distributed over a circumference of the tube (10), the improvement comprising: beginning at the distal end (12) and extending in a direction of the proximal end (11) the feeding probe (1) having a guide portion (F), the guide portion (F) having a length (L) greater than a diameter (D) of an intestine of the patient into which the distal end (12) of the tube (10) of the feeding probe (1) is introduced, the guide portion (F) having a length (L) of at least 30 mm, and the outlet openings (120) on the circumference of the tube (10) being disposed outside of the guide portion (F).

2. The feeding probe in accordance with claim 1, wherein the guide portion (F) has a length (L) such that a ratio between the diameter (D) of the intestine of the patient and the length (L) is at least 1:1.2.

3. The feeding probe in accordance with claim 2, wherein the guide portion (F) has a length (L) of 40 to 60 mm.

4. The feeding probe in accordance with claim 3, wherein the outlet openings (120) closest to the distal end (12) of the tube (10) at the circumference of the tube (10) have a minimum spacing of 30 mm from the distal end (12) of the tube (10) of the feeding probe (1).

5. The feeding probe in accordance with claim 4, wherein the tube (10) has a constant outside diameter (ADE) over a tube length of the tube (10).

6. The feeding probe in accordance with claim 5, wherein a second diameter (DA) of the distal outlet opening (121) is equivalent to a third diameter (DL) of the lumen (100) embodied in the tube (10), and peripheral regions (122) of the tube (10) surrounding the distal outlet opening (121) are rounded.

7. The feeding probe in accordance with claim 6, wherein the tube (10) is produced from a soft elastic material with high restoring force, including at least one of a thermoplastic, an elastomeric plastic, a thermoplastic elastomer, and an elastomer-like thermoplastic, with a Shore hardness A, in accordance with ASTM D 2240 of less than or equal to 90.

8. The feeding probe in accordance with claim 7, wherein the feeding probe is equipped to be radiopaque.

9. The feeding probe in accordance with claim 8, wherein the feeding probe contains from 10 to 15 parts by weight of a radiopaque medium per 100 parts by weight of the soft elastic plastic.

10. The feeding probe in accordance with claim 9, wherein a surface of the tube (10) is microscopically rough.

11. The feeding probe in accordance with claim 10, wherein the proximal end (11) of the tube (10) is connected to an adapter (110) for connection to a source of one of a feeding solution and a medication solution.

12. The feeding probe in accordance with claim 11, for an use wherein the feeding probe is used in conjunction with a gastric probe (5) for draining gastric secretions and decompression of a stomach of a patient.

13. The use in accordance with claim 12, wherein the gastric probe (5) has a second tube (50) with a second lumen (54) that is continuous from a second proximal end (51) to a second distal end (52) and has drainage holes (53) emerging laterally from the second tube (50), and the feeding probe (1) can be passed through the second lumen (54) of the second tube (50) of the gastric probe (5) forming an annular gap between a second surface of the feeding probe (1) and the inner layer which defines the second lumen (54) of the gastric probe (5) of the second tube (50).

14. The use in accordance with claim 13, wherein with the gastric probe (5) positioned over the feeding probe (1) the second tube (50) is multi-layer, with an outer layer (501) of a soft flexible plastic and an inner layer (502) of a harder plastic than the soft flexible plastic, and the second distal end (52, 52a) of the gastric probe (5) inside the passable second lumen (54, 54a) of the gastric probe (5), and the harder plastic of the inner layer (502) has a good sliding property and anti-adhesion property relative to a plastic of the feeding probe (1).

15. The use in accordance with claim 14, wherein the outer layer (501) is of at least one of a thermoplastic, an elastomeric plastic, a thermoplastic elastomer, and an elastomer-like thermoplastic, with a Shore hardness A in accordance with ASTM D 2240 of less than or equal to 90 and an inner layer of a plastic with a Shore hardness D in accordance with ASTM 2240 of greater than 45, and a thickness of the inner layer (502) of the second tube (50) is less than 0.020 mm.

16. The use in accordance with claim 15, wherein the second tube (50) is multi-layered and the second distal end (52) is formed by an atraumatic radiopaque probe tip (55) with the second lumen (54a) being continuous.

17. The use in accordance with claim 16, wherein each of the tube (10) of the feeding probe (1) and the outer layer (501) of the second tube (50) of the gastric probe (5) and the probe tip (55) of the gastric probe (5) is of a soft flexible plastic with a Shore hardness A of less than or equal to 90.

18. The use in accordance with claim 17, wherein a thermoplastic polyether urethane is used for the tube (10) of the feeding probe (1), the outer layer (501), and the probe tip (55) of the gastric probe (5), and a polyether block amide is used for the inner layer (502) of the second tube (50) of the gastric probe (5).

19. The use in accordance with claim 18, wherein a Y connector having three outlets with a first outlet connected to the second proximal end (51) of the second tube (50) of the gastric probe (5) and a second outlet (62) connected to a suction line (9) for the gastric probe (5), and a third outlet (61) connected to the feeding probe (1) which is guided through the second lumen (54, 54a) of the gastric probe (5) and can be extended to an outside.

20. The use in accordance with claim 19, wherein the feeding probe (1) is of 9 CH in size and has a length of at least 2 m, the gastric probe (5) is of 16 CH in size and has a length of at least 1 m that can be inserted via the feeding probe (1), and the continuous second lumen (54, 54a) of the gastric probe (5) has an inside diameter of at least 4 mm.

21. The feeding probe in accordance with claim 1, wherein the guide portion (F) has a length (L) of 40 to 60 mm.

22. The feeding probe in accordance with claim 1, wherein the outlet openings (120) closest to the distal end (12) of the tube (10) at the circumference of the tube (10) have a minimum spacing of 30 mm from the distal end (12) of the tube (10) of the feeding probe (1).

23. The feeding probe in accordance with claim 1, wherein the tube (10) has a constant outside diameter (ADE) over a tube length of the tube (10).

24. The feeding probe in accordance with claim 1, wherein a second diameter (DA) of the distal outlet opening (121) is equivalent to a third diameter (DL) of the lumen (100) embodied in the tube (10), and peripheral regions (122) of the tube (10) surrounding the distal outlet opening (121) are rounded.

25. The feeding probe in accordance with claim 1, wherein the tube (10) is produced from a soft elastic material with high restoring force, including at least one of a thermoplastic, an elastomeric plastic, a thermoplastic elastomer, and an elastomer-like thermoplastic, with a Shore hardness A, in accordance with ASTM D 2240 of less than or equal to 90.

26. The feeding probe in accordance with claim 1, wherein the feeding probe is equipped to be radiopaque.

27. The feeding probe in accordance with claim 26, wherein the feeding probe contains from 10 to 15 parts by weight of a radiopaque medium per 100 parts by weight of the soft elastic plastic.

28. The feeding probe in accordance with claim 1, wherein a surface of the tube (10) is microscopically rough.

29. The feeding probe in accordance with claim 1, wherein the proximal end (11) of the tube (10) is connected to an adapter (110) for connection to a source of one of a feeding solution and a medication solution.

30. The feeding probe in accordance with claim 1, for an use wherein the feeding probe is used in conjunction with a gastric probe (5) for draining gastric secretions and decompression of a stomach of a patient.

31. The use in accordance with claim 30, wherein the gastric probe (5) has a second tube (50) with a second lumen (54) that is continuous from a second proximal end (51) to a second distal end (52) and has drainage holes (53) emerging laterally from the second tube (50), and the feeding probe (1) can be passed through the second lumen (54) of the second tube (50) of the gastric probe (5) forming an annular gap between a second surface of the feeding probe (1) and the inner layer which defines the second lumen (54) of the gastric probe (5) of the second tube (50).

32. The use in accordance with claim 30, wherein with the gastric probe (5) positioned over the feeding probe (1) a second tube (50) is multi-layer, with an outer layer (501) of a soft flexible plastic and an inner layer (502) of a harder plastic than the soft flexible plastic, and a second distal end (52, 52a) of the gastric probe (5) inside a passable second lumen (54, 54a) of the gastric probe (5), and the harder plastic of the inner layer (502) has a good sliding property and anti-adhesion property relative to a plastic of the feeding probe (1).

33. The use in accordance with claim 30, wherein an outer layer (501) of a second tube (50) is of at least one of a thermoplastic, an elastomeric plastic, a thermoplastic elastomer, and an elastomer-like thermoplastic, with a Shore hardness A in accordance with ASTM D 2240 of less than or equal to 90 and an inner layer of a plastic with a Shore hardness D in accordance with ASTM 2240 of greater than 45, and a thickness of an inner layer (502) of the second tube (50) is less than 0.020 mm.

34. The use in accordance with claim 33, wherein the second tube (50) is multi-layered and the second distal end (52) is formed by an atraumatic radiopaque probe tip (55) with the second lumen (54a) being continuous.

35. The use in accordance with claim 34, wherein each of the tube (10) of the feeding probe (1) and the outer layer (501) of the second tube (50) of the gastric probe (5) and the probe tip (55) of the gastric probe (5) is of a soft flexible plastic with a Shore hardness A of less than or equal to 90.

36. The use in accordance with claim 35, wherein a thermoplastic polyether urethane is used for the tube (10) of the feeding probe (1), the outer layer (501), and the probe tip (55) of the gastric probe (5), and a polyether block amide is used for the inner layer (502) of the second tube (50) of the gastric probe (5).

37. The use in accordance with claim 30, wherein a Y connector having three outlets with a first outlet connected to the second proximal end (51) of the second tube (50) of the gastric probe (5) and a second outlet (62) connected to a suction line (9) for the gastric probe (5), and a third outlet (61) connected to the feeding probe (1) which is guided through a second lumen (54, 54a) of the gastric probe (5) and can be extended to an outside.

38. The use in accordance with claim 30, wherein the feeding probe (1) is of 9 CH in size and has a length of at least 2 m, the gastric probe (5) is of 16 CH in size and has a length of at least 1 m that can be inserted via the feeding probe (1), and a continuous second lumen (54, 54a) of the gastric probe (5) has an inside diameter of at least 4 mm.
